(19) United States
(12) Patent Application Publication
(10) Pub. No.: US 2021/0282930 A1
(43) Pub. Date: Sep. 16, 2021

(54) BONE TRABECULA STRUCTURE AND PROSTHESIS USING SAME AND MANUFACTURING METHOD THEREFOR

(71) Applicant: BEIJING CHUNLIZHENGDA MEDICAL INSTRUMENTS CO., LTD, Beijing (CN)

(72) Inventors: Chunbao SHI, Beijing (CN); Kuixue XU, Beijing (CN); Xiaoqiang LU, Beijing (CN); Fengbao XIE, Beijing (CN); WANG Zhenguo, Beijing (CN); Zeyu DONG, Beijing (CN); Wenchao SHI, Beijing (CN)

(21) Appl. No.: 17/257,534

(22) PCT Filed: Nov. 20, 2019
(86) PCT No.: PCT/CN2019/119619

§ 371 Date: Dec. 31, 2020

(30) Foreign Application Priority Data
Dec. 4, 2018 (CN) 201811475455.3

(51) Int. Cl. A61F 2/28; A61F 2/30

(52) U.S. Cl. CPC A61F 2/28; A61F 2/30942; A61F 2/30767; A61F 2002/30024; A61F 2002/3092; A61F 2002/30985; A61F 2310/00023; A61F 2002/30948

(57) ABSTRACT

A bone trabecular structure, a prosthesis having the same, and a fabrication method thereof are provided. The bone trabecular structure includes a body configured to be a three-dimensional porous structure, which includes a plurality of struts and a plurality of pores formed by staggered connection of the plurality of struts. The pores are communicated with each other and have different average diameters. The average diameter of the pores ranges from 100 μm to 400 μm, and the porosity of the three-dimensional porous structure ranges from 50% to 80%. This bone trabecular structure facilitates postoperative bone ingrowth of a patient, and thus effectively improves the postoperative recovery effect of the patient.

### CROSS-REFERENCE TO RELATED APPLICATIONS

This application is a national stage application of PCT/CN2019/119619. This application claims priorities from PCT Application No. PCT/CN2019/119619, filed Nov. 20, 2019, and claims the priority benefit of China patent application No. 201811475455.3 titled “Bone Trabecular Structure And Prosthesis Having The Same” filed on Dec. 4, 2018 by Beijing Chunlizhengda Medical Instruments Co., Ltd. The entirety of the above-mentioned patent applications are hereby incorporated by reference herein.

### FIELD OF THE INVENTION

The invention relates to the field of medical prosthesis, and, in particular, to a bone trabecular structure, a prosthesis having the same and a fabrication method thereof.

### DESCRIPTION OF RELATED ART

In a surgical operation, when tumor, nonunion after fracture or other pathological changes in the limb backbone of a human body lead to partial bone defects or surgical resections, it is necessary to replace them with an artificial prosthesis. In the operation of prosthesis replacement, an artificial prosthesis have to be connected and fused with bone tissues to restore the physiological function of the limb backbone.

At present, most of the existing prosthesis are cemented prosthesis and titanium-coated cementless prosthesis. Cemented prosthesis may suffer from a greater risk of prosthesis loosening in the later stage, which will lead to surgical failure. However, titanium-coated prosthesis will produce metal corrosion in complex human body environment, resulting in the release of toxic elements and thus reduced biocompatibility. In addition, the elastic modulus of the metal material is quite different from that of human bone tissues, which is easy to produce stress shielding effect, which is not conducive to the growth and remodeling of new bones, and even leads to secondary fracture. Moreover, the repair of bone defects caused by bone trauma and necrosis has poor mechanical and osteoinductive properties.

In order to improve the bone ingrowth effect after prosthesis implantation, a bone trabecular structure is increasingly applied to an artificial prosthesis in the field. The bone trabecular structure is mainly used to make the prosthesis form a number of pores conducive to bone ingrowth, so as to improve the stability of the prosthesis after implantation. However, the existing bone trabecular structure usually has uniform and equal diameter distribution, and lacks bionic characteristics of the real structure of a human body. In the actual service process after implantation, the structural strength of some parts is insufficient due to the uneven stress characteristics of the human body, while the structural strength of other parts suffers from over designing. If the bone trabecular structure is not in the best condition with the joint face of the bone, the early stability of the prosthesis will be affected, and then the growth of the bone will be affected, which is not conducive to long-term and stable prosthesis service.

In view of the deficiencies present in the prior art, those skilled in the art urgently hope to seek a bone trabecular structure which is more conducive to bone ingrowth and improve the postoperative recovery effect, so as to address the deficiencies present in the prior art.

### BRIEF SUMMARY OF THE INVENTION

In order to facilitate bone ingrowth and improve the postoperative recovery effect of a patient, the present application provides a bone trabecular structure, a prosthesis having the same and a fabrication method thereof.

According to a first aspect of the present application, a bone trabecular structure is provided, which includes a body configured to be a three-dimensional porous structure. The three-dimensional porous structure includes a plurality of struts and a plurality of pores formed by staggered connection of the plurality of struts. The pores are communicated with each other, and the average diameters of the pores are different from each other. In particular, the average diameter of the pores ranges from 100 μm to 400 μm, and the porosity of the three-dimensional porous structure ranges from 50% to 80%.

Further, the three-dimensional structure includes a plurality of regions adapted to different growth requirements of the same tissue, and the porosity of the regions is different from each other.

Further, in the same region, the density of pores in the direction from the outside to the inside of the three-dimensional porous structure is increased gradually.

The diameter of the struts ranges from 100 μm to 200 μm.

Further, the cross-section shape of the pores is irregular polygon.

Further, a plurality of convex portions are formed on the peripheral walls of the struts.

Further, the three-dimensional porous structure is made of titanium alloy material.

Further, the elastic modulus of the three-dimensional porous structure ranges from 5-30 GPa.

Further, the maximum static friction coefficient of the outer surface of the three-dimensional porous structure ranges from 1.2 to 1.5.

According to a second aspect of the present application, a prosthesis is provided. The prosthesis includes a prosthesis body and the above bone trabecular structure formed on an outer surface of the prosthesis body.

According to a third aspect of the present application, a fabrication method of a bone trabecular structure is provided, which includes the following steps:

Step 1: scanning a natural bone trabecular structure by a Micro CT, and remodeling the scanned data by using MIMICS to obtain a three-dimensional schematic model of the bone trabecular structure. The Step 1 is used for obtaining the basic structure model of the bone trabecular structure in advance;

Step 2: adjusting the diameter of the struts in the three-dimensional schematic model of the bone trabecular structure to be between 100 μm and 200 μm, and adjusting the diameter of the pores formed by the struts so that the average diameter range of the pores ranges from 100 μm to 400 μm, and the porosity of the three-dimensional schematic model ranges from 50% to 80%;

Step 3: dividing the three-dimensional schematic model of the bone trabecular structure into different regions which are respectively adapted for different growth requirements of the same tissue, and further adjusting the diameter of the pores in the regions so that different regions have different porosity, whereby a bone tissue can grow into the bone trabecular structure more quickly and adaptively under different bone growth requirements;

Step 4: generating a solid model of the bone trabecular structure by using a 3D printing equipment, in which the focus offset parameter of the 3D printing device is adjusted so that a plurality of bumps are formed on the surface of the struts in the generated solid model. The value of the focus offset parameter ranges from 5.8 mA to 6.2 mA.

Further, the porosity of the regions divided in Step 3 is different from each other.

Further, in the same region, the density of pores in the direction from outside to inside is increased gradually.

Further, the cross-section shape of the pores is irregular polygon.

Further, a plurality of convex portions are formed on the peripheral wall of the struts.

Further, the struts and the pores form a three-dimensional porous structure which is made of titanium alloy material.

Further, the elastic modulus of the three-dimensional structure ranges from 5 to 30 GPa

Further, the maximum static friction coefficient of the outer surface of the three-dimensional porous structure ranges from 1.2 to 1.5.

In the bone trabecular structure of the present application, the pores formed by staggered connection of the struts are communicated with each other, the average diameter of the pores is configured to be different from each other, and the average diameter and porosity of the pores of the three-dimensional porous structure are specifically set, so that the structure of the bone trabecular structure more resembles that of the bone trabecular structure in a human body, and the bone of the human body can grow into the pores of the three-dimensional porous structure rapidly and naturally, and facilitate a quick postoperative fusion and fixation of the bone trabecular structure with the bone tissues of the human body. Therefore, it can effectively improve the postoperative recovery effect of a patient.

### BRIEF DESCRIPTION OF DRAWINGS

In order to more clearly explain technical solutions provided the Detailed Description of the present application or in the prior art, drawings needed for describing Detailed Description or the prior art will be briefly described. In all drawings, similar elements or parts are generally identified by similar reference signs. In the drawings, the elements or parts are not necessarily drawn to actual scale.

FIG. 1 is an enlarged perspective view of part of the trabecular structure according to the present application;

FIG. 2 is a schematic diagram of the main cross section of the trabecular structure shown in FIG. 1; and

FIG. 3 is a flow chart of the fabrication method of the bone trabecular structure according to the present application.

### DETAILED DESCRIPTION

Embodiments of the technical solutions of the present application will be described in detail in connection with the drawings. The following embodiments are merely provided for explaining the technical solutions of the present application more clearly, and thus is only used as an example, without any intention to limit the protection scope of the present application.

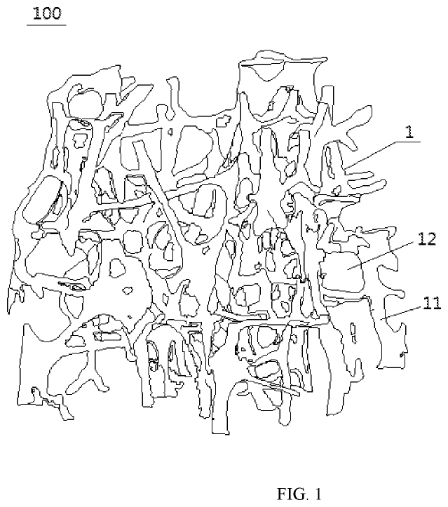

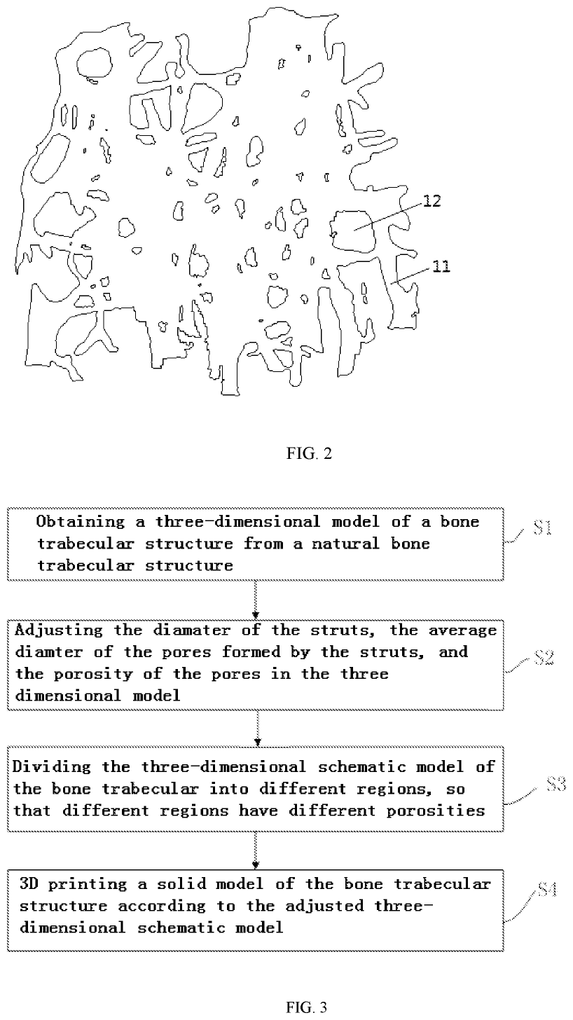

FIG. 1 shows the structure of a bone trabecular structure 100 according to the present application. The bone trabecular structure 100 includes a body configured to be a three-dimensional porous structure 1. The three-dimensional porous structure 1 includes a plurality of struts 11 and a plurality of pores 12 formed by staggered connection of the plurality of struts 11. The pores 12 are communicated with each other, and the average diameter of the pores 12 is different from each other. In particular, the average diameter of the pores 12 ranges from 100 μm to 400 μm, and the porosity of the three-dimensional porous structure ranges from 50% to 80%.

In the bone trabecular structure 100 of the present application, the pores 12 formed by staggered connection of the struts 11 are communicated with each other, the average diameter of the pores 12 is configured to be different from each other, and the average diameter and porosity of the pores 12 of the three-dimensional porous structure 1 are specifically set, so that the structure of the bone trabecular structure 100 more resembles that of the bone trabecular structure in a human body, and the bone of the human body can grow into the pores 12 of the three-dimensional porous structure 1 rapidly and naturally, and facilitate a quick postoperative fusion and fixation of the bone trabecular structure 100 with the bone tissues of the human body. Therefore, it can effectively improve the postoperative recovery effect of a patient.

In a preferred embodiment, the three-dimensional porous structure 1 may include a plurality of regions adapted to different growth requirements of the same tissue, and the porosity of individual regions is different from each other. That is, in different regions of the three-dimensional porous structure 1, regarding different bone ingrowth requirements, the porosity of different regions can be set specifically. The porosity of different regions can be set by controlling the average diameter of the pores in corresponding regions. For example, when some region of the bone trabecular structure 100 needs to contact with bone, it is preferred to set the average diameter range of pores in this region between 200 μm and 400 μm, and further preferably between 300 μm and 400 μm in order to achieve rapid bone ingrowth for which the porosity in this region can be controlled between 50% and 60%. When some region of the bone trabecular structure 100 does not need to contact with bone, but needs to facilitate bone crawling, the range of average diameter of pores in this region can be set between 100 μm and 200 μm for which the porosity of this region can be controlled between 70% and 75%. When some region of the trabecular structure 100 neither needs to contact with bone nor needs to facilitate bone crawling, the average diameter range of pores in this region can be set between 100 μm and 150 μm for which the porosity of this region can be controlled between 75% and 80%.

Preferably, in the same region, the density of the pores 12 in the direction from the outside to the inside of the three-dimensional porous structure 1 gradually increases, so that the bone tissues can grow more smoothly and quickly from the outside of the three-dimensional porous structure 1 into the internal center of the three-dimensional porous structure 1, so as to improve the fusion and fixation of the bone trabecular structure 100 with human bone tissues.

Further preferably, in order to ensure the structural strength of the three-dimensional porous structure 1, the diameter range of the struts 11 can be set between 100 μm and 200 μm. Preferably, the diameter range of the struts 11 is set between 150 μm and 200 μm, and further preferably, the diameter of the struts 11 is 180 μm.

As shown in FIG. 1, the cross-sectional shape of the pores 12 of the three-dimensional porous structure 1 is irregular polygon, and its particular shape can be roughly tetrahedron or hexahedron.

In a preferred embodiment, a plurality of convex portions (not shown) can be formed on the peripheral wall of the struts 11. The providing of the plurality of convex portions is used to increase the roughness of the peripheral wall of the struts 11, so as to increase the friction force of the peripheral wall. In this way, the fixation of bone tissues with strut 11 is more firm and stable during bone ingrowth.

Preferably, the convex portion can be a circular convex point or a conical convex point. It should be noted that the shape of the convex portion is not limited to the above-mentioned shapes, as long as the convex portion can effectively increase the external surface area of the struts 11 to increase the friction force of its peripheral wall, and thus will not be described here in detail. Further, the width range of the maximum profile shape of the cross section of the convex portion is 5-50 μm and the maximum height range is 10-50 μm.

Further preferably, the convex portions can be evenly distributed on the outer surface of the struts 11, or can be distributed in a discrete manner. Further preferably, the number of convex portions of the struts 11 in a region of the bone trabecular structure 100 having a higher porosity may be greater than the number of the convex portions of the struts 11 in a region of the bone trabecular structure 100 having a lower porosity, so as to facilitate firmer bone ingrowth. It is further preferred that the density of the convex portions on the struts 11 at an outer edge of the three-dimensional porous structure 1 is greater than that of the convex portions on the struts 11 at the interior of the three-dimensional porous structure 1.

According to the present application, the three-dimensional porous structure 1 can be made of metal powder, which can be titanium alloy, pure titanium or tantalum metal, etc. Preferably, the three-dimensional porous structure 1 is made of titanium alloy material, preferably made of Ti6Al4V.

According to the present application, the elastic modulus of the three-dimensional porous structure 1 ranges from 5 to 30 GPa. This range of the elastic modulus can provide the bone trabecular structure 100 with better mechanical properties, that is, better compression and torsion resistance, so as to ensure the stability of the bone trabecular structure 100 after implantation into a human body.

Further, the maximum static friction coefficient of the outer surface of the three-dimensional porous structure 1 ranges from 1.2 to 1.5. In this range, the external surface of the bone trabecular structure 100 has a roughness more suitable for bone growth or crawling, thus facilitating further improving the speed and stability of bone ingrowth therein. Meanwhile, compared with a bone trabecular structure in the prior art, the maximum static friction force of the external surface of the bone trabecular structure 1 in the present application is far greater than that of the bone trabecular structure in the prior art, so that the bone trabecular structure 100 of the present application significantly differs from those in the prior art, and the bone trabecular structure 100 of the present application provide more suitable conditions for human bone growth and bone crawling. Upon testing, it is shown that, under the same testing conditions, the maximum static friction coefficient of the bone trabecular structure 100 of the present application is 1.35 under unit pressure, while the maximum static friction coefficient of the bone trabecular structure 100 in the prior art under unit pressure is only 1.08.

In addition, the present application provides a prosthesis (not shown). The prosthesis includes a prosthesis body and the above-mentioned bone trabecular structure 100 formed on the outer surface of the prosthesis body. Since the bone trabecular structure 100 of the present application more resembles the bone trabecular structure of a human body, the bone of the human body can quickly and naturally grow into the pores 12 of the three-dimensional porous structure 1, which is conducive to a rapid fusion and fixation of the prosthesis with the bone tissues of the human body after an operation, thereby effectively improving the postoperative recovery effect of a patient.

FIG. 3 shows a flow chart of a fabrication method of a bone trabecular structure according to the present application. As shown in FIG. 3, the fabrication method of the bone trabecular structure includes the following steps:

Step 1: scanning a natural bone trabecular structure by a Micro CT, and remodeling the scanned data by using MIMICS to obtain a three-dimensional schematic model of the bone trabecular structure. This step is used for obtaining the basic structure model of the bone trabecular structure in advance.

Step 2: adjusting the diameter of the struts 11 in the three-dimensional schematic model of the bone trabecular structure to be between 100 μm and 200 μm, and adjusting the diameter of the pores 12 formed by the struts 11 so that the average diameter of the pores 12 ranges from 100 μm to 400 μm, and the porosity of the three-dimensional schematic model ranges from 50% to 80%.

Step 3: dividing the three-dimensional schematic model of the bone trabecular structure into different regions which are respectively adapted for different growth requirements of the same tissue, and further adjusting the diameter of the pores 12 in the regions so that different regions have different porosity, whereby a bone tissue can grow into the bone trabecular structure more quickly and adaptively under different bone growth requirements.

Step 4: generating a solid model of the bone trabecular structure 100 by using a 3D printing equipment, in which the focus offset parameter of the 3D printing device is adjusted so that a plurality of bumps are formed on the surface of the struts 11 in the generated solid model. In particular, the value of the focus offset parameter ranges from 5.8 mA to 6.2 mA.

Upon testing, it is shown that the bone trabecular structure 100 of the present application has good initial stability and good bone ingrowth performance, that is, the growing-in depth of the bone can reach 60% to 80%. The prosthesis using the bone trabecular structure 100 of the present application has a very stable effect after implantation, effectively reduces the postoperative recovery time of a patient, and improves the postoperative recovery effect of the patient.

It should be noted that, unless otherwise specified, the technical terms or scientific terms used in the present application shall have the common meaning understood by those skilled in the art.

Finally, it should be noted that, the above embodiments are merely provided for illustrating the technical solutions of the present application, without any limitation thereto. Although the invention has been described in detail with reference to the above-mentioned embodiments, those skilled in the art will understand that, the technical solutions recited in the above-mentioned embodiments can still be modified or some or all of the technical features therein can be equivalently substituted by those skilled in the art. These modifications or substitutions do not make the essence of the corresponding technical solutions depart from the scope of the technical solutions of the embodiments of the present application, which shall be covered in the scope of the claims and the description of the present application. In particular, as long as there is no structural conflict, the technical features mentioned in individual embodiments can be combined in any way. The invention is not limited to the specific embodiments disclosed herein, but includes all technical solutions falling within the scope of the claims.

1. A bone trabecular structure, comprising a body configured to be a three-dimensional porous structure comprising a plurality of struts and a plurality of pores formed by staggered connection of the plurality of struts, communicated with each other and having different average diameters, wherein the average diameter of the pores ranges from 100 μm to 400 μm, and the porosity of the three-dimensional porous structure ranges from 50% to 80%.

2. The bone trabecular structure according to claim 1, wherein the three-dimensional structure comprises a plurality of regions adapted to different growth requirements of the same tissue, and the porosity of the regions is different from each other.

3. The bone trabecular structure according to claim 2, wherein, in the same region, the density of pores in the direction from the outside to the inside of the three-dimensional porous structure is increased gradually.

4. The bone trabecular structure according to claim 1, wherein the diameter of the struts ranges from 100 μm to 200 μm.

5. The bone trabecular structure according to claim 1, wherein the cross-section shape of the pores is irregular polygon.

6. The bone trabecular structure according to claim 1, wherein a plurality of convex portions are formed on the peripheral walls of the struts.

7. The bone trabecular structure according to claim 1, wherein the three-dimensional porous structure is made of titanium alloy material.

8. The bone trabecular structure according to claim 7, wherein the elastic modulus of the three-dimensional porous structure ranges from 5-30 GPa.

9. The bone trabecular structure according to claim 1, wherein the maximum static friction coefficient of an outer surface of the three-dimensional porous structure ranges from 1.2 to 1.5.

10. A prosthesis, comprising a prosthesis body and the bone trabecular structure according to claim 1 formed on an outer surface of the prosthesis body.

11. A fabrication method of a bone trabecular structure comprising the following steps:
Step 1: scanning a natural bone trabecular structure by a Micro CT, and remodeling the scanned data by using MIMICS to obtain a three-dimensional schematic model of the bone trabecular structure, so as to obtain a basic structure model of the bone trabecular structure in advance;
Step 2: adjusting the diameter of the struts in the three-dimensional schematic model of the bone trabecular structure to be between 100 μm and 200 μm, and adjusting the diameter of the pores formed by the struts so that the average diameter range of the pores ranges from 100 μm to 400 μm, and the porosity of the three-dimensional schematic model ranges from 50% to 80%;
Step 3: dividing the three-dimensional schematic model of the bone trabecular structure into different regions which are respectively adapted for different growth requirements of the same tissue, and further adjusting the diameter of the pores in the regions so that different regions have different porosity, whereby a bone tissue can grow into the bone trabecular structure more quickly and adaptively under different bone growth requirements; and
Step 4: generating a solid model of the bone trabecular structure by using a 3D printing equipment, in which the focus offset parameter of the 3D printing device is adjusted, so that a plurality of bumps are formed on the surface of the struts in the generated solid model, wherein the value of the focus offset parameter ranges from 5.8 mA to 6.2 mA.

12. The fabrication method of a bone trabecular structure according to claim 11, wherein the porosity of the regions divided in Step 3 is different from each other.

13. The fabrication method of a bone trabecular structure according to claim 12, wherein, in the same region, the density of pores in the direction from outside to inside is increased gradually.

14. The fabrication method of a bone trabecular structure according to claim 11, wherein the cross-section shape of the pores is irregular polygon.

15. The fabrication method of a bone trabecular structure according to claim 11, wherein a plurality of convex portions are formed on the peripheral wall of the struts.

16. The fabrication method of a bone trabecular structure according to claim 11, wherein the struts and the pores form a three-dimensional porous structure which is made of titanium alloy material.

17. The fabrication method of a bone trabecular structure according to claim 16, wherein the elastic modulus of the three-dimensional structure ranges from 5 to 30 GPa.

18. The fabrication method of a bone trabecular structure according to claim 16, wherein the maximum static friction coefficient of an outer surface of the three-dimensional porous structure ranges from 1.2 to 1.5.

19. The bone trabecular structure according to claim 2, wherein a plurality of convex portions are formed on the peripheral walls of the struts.

20. The bone trabecular structure according to claim 3, wherein a plurality of convex portions are formed on the peripheral walls of the struts.
